(19) United States
(12) Patent Application Publication
(10) Pub. No.: US 2024/0310248 A1
(43) Pub. Date: Sep. 19, 2024

(54) Simultaneous Estimation of Water Encroachment and Bound Fluid in the Absence of NMR in Carbonate Reservoirs

(71) Applicant: Saudi Arabian Oil Company, Dhahran (SA)

(72) Inventors: Umar Idris, Dhahran (SA); Mustafa I. Abdulmohsin, Qatif (SA); Endurance O. Ighodalo, Dhahran (SA)

(21) Appl. No.: 18/122,024

(22) Filed: Mar. 15, 2023

(51) Int. Cl. G01M 99/00

(52) U.S. Cl. CPC G01M 99/005

(57) ABSTRACT

A computer-implemented method for simultaneous estimation of water encroachment and bound fluid in the absence of NMR in carbonate reservoirs is described. The bulk volume of water (BVW) is computed by multiplying porosity and water saturation. The Buckles number is computed for each petrophysical rock type (PRT). Water saturation in unseen wells is estimated using the Buckles number, porosity (from porosity logs) and PRT. Water saturation is evaluated to estimate water encroachment. Additionally, bound fluid for the new wells is estimated in the absence of NMR data.

### TECHNICAL FIELD

This disclosure relates generally to water encroachment and bound fluid estimation.

### BACKGROUND

Nuclear magnetic resonance (NMR) logging exploits the large magnetic moment of hydrogen, which is abundant in rocks in the form of water. NMR data is logged and can be used to determine various properties of interest, such as porosity, water content, water saturation, and permeability estimates of a formation. NMR logging is performed using highly sophisticated and expensive components.

### SUMMARY

An embodiment described herein provides a method for estimating water encroachment and bound fluid in the absence of nuclear magnetic resonance. The method includes computing, using at least one hardware processor, a bulk volume of water (BVW) per petrophysical rock type (PRT) by multiplying porosity and water saturation for respective PRT types across multiple wells. The method also includes computing, using the at least one hardware processor, a Buckles Number for the respective PRT types across multiple wells based on respective BVW. Additionally, the method includes estimating, using the at least one hardware processor, water saturation in unseen wells using the Buckles Number, porosity, and PRT, and simultaneously estimating bound fluid for the unseen wells using the PRT and the Buckles Number per PRT.

An embodiment described herein provides an apparatus comprising a non-transitory, computer readable, storage medium that stores instructions that, when executed by at least one processor, cause the at least one processor to perform operations. The operations include computing a bulk volume of water (BVW) per petrophysical rock type (PRT) type by multiplying porosity and water saturation for respective PRT types across multiple wells. The operations include computing a Buckles Number for the respective PRT types across multiple wells based on respective BVW. Additionally, the operations include estimating water saturation in unseen wells using the Buckles Number, porosity, and PRT, and simultaneously estimating bound fluid for the unseen wells using the PRT and the Buckles Number per PRT.

An embodiment described herein provides a system. The system comprises one or more memory modules and one or more hardware processors communicably coupled to the one or more memory modules. The one or more hardware processors is configured to execute instructions stored on the one or more memory models to perform operations. The operations include computing a bulk volume of water (BVW) per petrophysical rock type (PRT) type by multiplying porosity and water saturation for respective PRT types across multiple wells. The operations also include computing a Buckles Number for the respective PRT types across multiple wells based on respective BVW. Further, the operations include estimating water saturation in unseen wells using the Buckles Number, porosity, and PRT, and simultaneously estimating bound fluid for the unseen wells using the PRT and the Buckles Number per PRT.

In some embodiments, the water saturation is calculated using a water saturation model or a saturation height function.

In some embodiments, PRT types are determined using a rock quality value generated by a petrophysical rock type classification model.

In some embodiments, the rock quality value is associated with petrophysical rock types according to at least one range limit applied to the PRT.

In some embodiments, the petrophysical rock type classification model is a Flow Zone Indicator or Winland R35.

In some embodiments, the Buckles Number is calculated from a histogram of the BVW per PRT type for the multiple wells.

In some embodiments, the porosity for the unseen wells is obtained from log data.

### BRIEF DESCRIPTION OF DRAWINGS

FIG. 1 is a workflow that shows data preparation for the estimation of water encroachment and bound fluid in the absence of NMR in carbonate reservoirs.

FIG. 2 shows a Buckles Number determination per PRT.

FIG. 3 is an illustration of a PRT types of a section of a well.

FIG. 4 shows the Buckles Number per PRT.

FIG. 5 shows a Buckles plot with mean Buckles Number clusters for respective PRT.

FIG. 6 is a workflow for determining the Buckles Number.

FIG. 7 is a process flow diagram of a process for the estimation of water encroachment and bound fluid in the absence of NMR in carbonate reservoirs using petrophysical rock type and Buckles Number.

FIG. 8 is a schematic illustration of an example controller 800 (or control system) for the estimation of water encroachment and bound fluid in the absence of NMR in carbonate reservoirs using petrophysical rock type and Buckles Number according to the present disclosure.

### DETAILED DESCRIPTION

Accurate determination of water saturation in a producing reservoir is essential for planning infill development wells. In examples, infill wells are drilled to replace reserves as parent well production declines. For example, infill wells are placed in a field to decrease average well spacing. The addition of infill wells accelerates expected recovery and increases estimated ultimate recovery in heterogeneous reservoirs. As well spacing is decreased, the shifting well patterns alter the formation-fluid flow paths and increase sweep to areas where greater hydrocarbon saturations exist.

Water saturation can be computed using Archie equations. The Archie equations determine water saturation based on parameters such as porosity, formation water resistivity, observed bulk resistivity, cementation exponent, and a saturation exponent. Porosity refers to pore volume, void space, or a volume within rock that can contain fluid. Formation water resistivity is a function of water salinity and temperature. Observed bulk resistivity refers to the ability of a material to resist electrical conduction. The cementation exponent represents insulating minerals that reduce the conductivity of the formation fluid, while the saturation exponent represents the effect of desaturating the sample or replacing of formation water with non-conductive hydrocarbons. The cementation and saturation exponents are known from core data, and the accuracy of the resulting water saturation calculation depends on the accuracy of the Archie parameters. In a reservoir undergoing water injection, formation water resistivity becomes uncertain due to the mixture of the formation water and the injection water. In examples, water injection is the injection of water into the reservoir to pressurize and displace hydrocarbons to producing wells, while formation water is water that occurs naturally within the pores of rock. Because of the mixed water salinity between the formation water and the injection water, the computed water saturation becomes highly uncertain. Nuclear Magnetic Resonance (NMR) logging can be performed to record NMR logs that include downhole formation samples in every lobe of the reservoir, and the NMR logs can be used to validate the computed water saturation. NMR techniques are expensive, complex, and frequently require supplementary and expensive non-NMR measurements. Further, NMR techniques are intrinsically insensitive when compared with other modalities due to the sparse, insufficiently concentrated samples available with NMR logging.

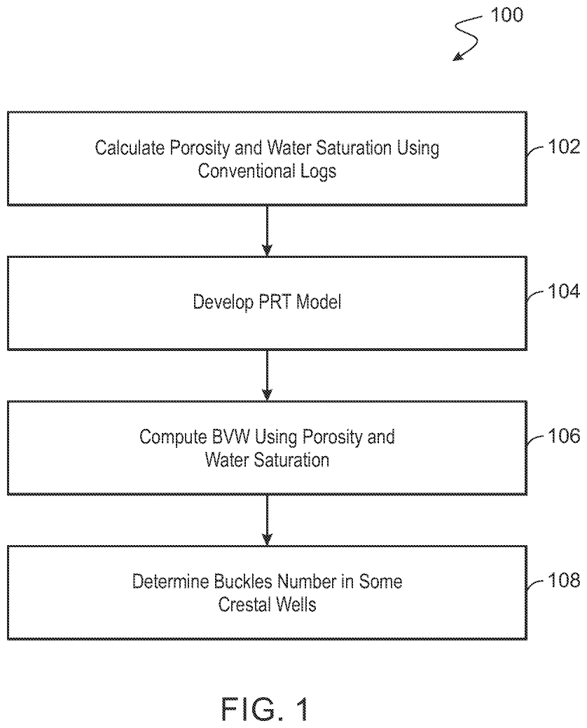

The present techniques enable the estimation of water encroachment and bound fluid in the absence of NMR in carbonate reservoirs. In examples, a petrophysical rock type and Buckles Number are used to simultaneously estimate water encroachment and bound fluid in the absence of NMR data. FIG. 1 is a workflow 100 that shows data preparation for the estimation of water encroachment and bound fluid in the absence of NMR in carbonate reservoirs. At block 102, porosity and water saturation is calculated using well logs. In examples, data is captured as a suite of logging measurements recorded as logging tools travel across an interval of a well, defined by depth. The well logs include the magnitude of a specific formation property, such as spontaneous potential, gamma ray, resistivity, neutron and density curves. Accordingly, data captured in logs such as gamma ray, resistivity, density, neutron, sonic logs are used to calculate porosity and water saturation. In examples, the well logs are first interpreted based on a resistivity interpretation. In a resistivity interpretation of the log data, depth intervals with a high resistivity represent hydrocarbons, while depth intervals with a low resistivity represent water. An exception to this resistivity interpretation is if there is change in texture or low resistivity pay. For example, a high resistivity indicates very tight carbonate, anhydrite, or rock that is relatively non-conductive. High-resistivity can indicate the presence of low-salinity fluids or hydrocarbons, which have a lower conductivity than brine. A low resistivity indicates the rock is relatively conductive, and can indicate the presence of high-conductivity fluids or minerals. Low resistivity can be an indicator of the presence of water bearing formations in a reservoir. In examples, water saturation is computed from resistivity and porosity logs using any of the resistivity-based saturation models or a saturation height function.

In a first example, porosity and water saturation (S_{w}) are computed with available porosity logs, using a water saturation model. The water saturation model is, for example, based on equations including but not limited to Archie, Waxman-Smits, Dual Water Equations, Simandoux, Indonesia, Fertl, S_{w} ratio, or any combinations thereof. In a second example, porosity and water saturation (S_{w}) are computed from a saturation height function (SHF) built from core measured capillary pressure data. In intervals where the log-derived S_{w} and SHF derived S_{w} match, the interval is still at irreducible water saturation (SWIRR) (assuming the water saturation model and SHF are correct).

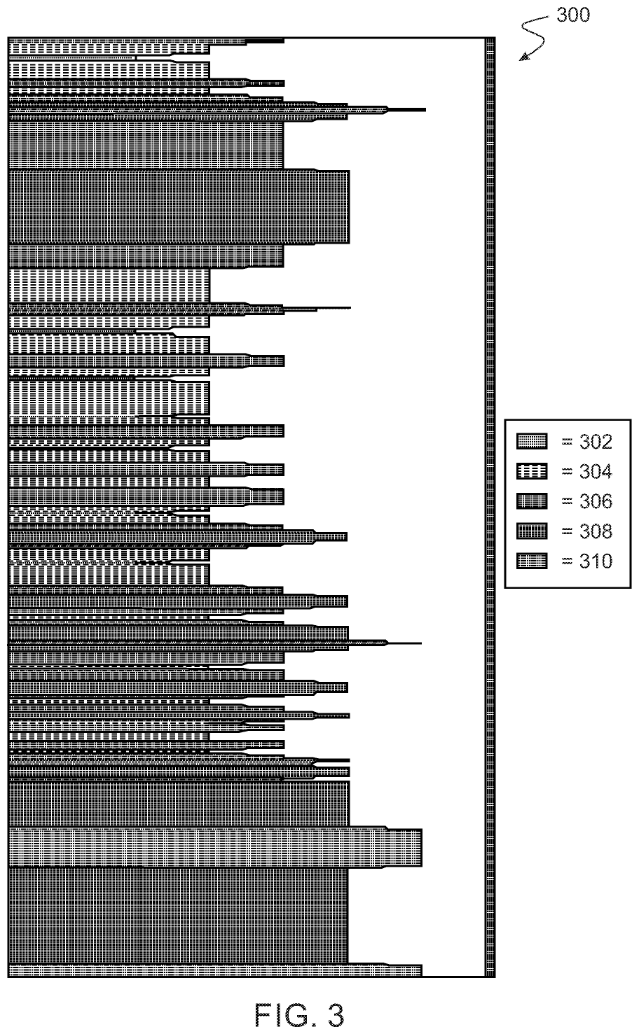

At block 104, at least one PRT model is developed by determining the petrophysical rock types based on core data of the logged wells. FIG. 3 is an illustration of a PRT types 300 of a section of a well. The PRT types 300 includes PRTs 302, PRT 304, PRT 306, PRT 308, and PRT 310. A PRT is a representative reservoir unit with a distinct porosity-permeability relationship and a unique water saturation for a given height above free water level. In examples, a PRT model is developed for cored wells, and the developed PRT model is applied to other wells to output a PRT type based on the logged data.

PRT models are developed, respectively, using logs and core data as input to determine rock quality values. In embodiments, rock types correspond to a distribution of the rock quality values for the core samples. The rock quality values are grouped into similar pore types or over-all rock qualities. The grouping can include generating a histogram of the distribution of the rock quality values and segmenting the distribution into different groups of rock quality values. The segmenting may be conducted by way of various petrophysical rock type classification techniques. In examples, the segmenting is performed according to Flow Zone Indicator (FZI) or Winland R35 (R35) techniques. Details regarding the FZI method are described in Rebelle M. and Lalanne B., 2014, Rock-Typing In Carbonates: A Critical Review Of Clustering Methods, SPE-171759-MS, which is incorporated by reference. Details regarding the Winland R35 method are described by Kolodzie, S. I, 1980, The analysis of Pore throat Size and Use of Waxman-Smit to Determine OOIP in Spindle Field, Colorado: SPE 55th Annual Fall Tech. Conf. and Exhib., SPE paper 0382), which is incorporated by reference.

The Flow Zone Indicator (FZI) techniques determine intervals of rock types that maintain the geologic framework and share rock type characteristics. Data used for computing FZI includes porosity and permeability, either core porosity and core permeability (for calibration), or log derived porosity and log derived permeability. The FZI equation relating porosity and permeability is as follows:

$$R Q I = 0.0314 * √ permeability / porosity \qquad ( 1 )$$

$$PHIZ = porosity / ( 1 - porosity ) \qquad ( 2 )$$

$$FZI = R Q I / PHIZ \qquad ( 3 )$$

where RQI is the rock quality indicator, PHIZ is the porosity factor, and FZI is the rock quality value.

Winland R35 techniques determine a rock quality value using Winland empirical equations that relate porosity, permeability and pore aperture based on mercury injection capillary pressure data. The Winland equation relating porosity and permeability is as follows:

$$LogR 35 = 0.732 + 0.588 * log ( Permeability ) - 0.864 * Log ( Porosity ) \qquad ( 4 )$$

where R35 is the rock quality value.

The resultant rock quality values are assigned to petrophysical rock types observed in the core data to build the PRT model. In examples, each petrophysical rock type is associated with a range of rock quality values. The PRT model is applied to wells without core data to generate rock quality values based on log data (e.g., permeability logs, porosity logs, etc.). The rock quality values are classified into a PRT based on the predetermined rock property ranges associated with each PRT.

Referring again to FIG. 1, at block 106, the bulk volume of water (BVW) is calculated using the porosity and the water saturation of the PRTs. The BVW is calculated by multiplying porosity and water saturation. The water saturation is derived from a saturation height function or obtained from pre-production crestal wells that are at irreducible water saturation. In examples, crestal wells are drilled on the crest or near the crest of the formation. These are wells on crest of the structure and far from the free-water-level (FWL) or oil-water-contact (OWC).

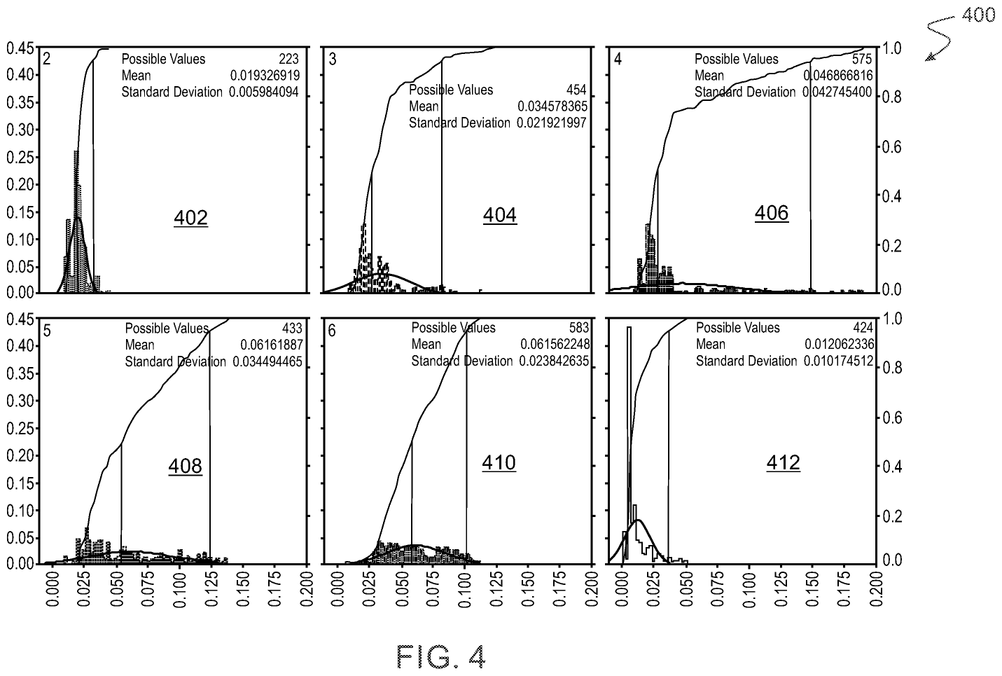

At block 108, the Buckles Number is determined in some crestal wells. The Buckles Number is the mean BVW for the PRTs of a respective well. FIG. 4 shows the Buckles Number per PRT. In the example of FIG. 4, each plot is a histogram of the Buckles Number per PRT type, and includes a contour that represents equal bulk volume water. The histograms 402, 404, 406, 408, and 410 correspond to the PRTs 302, 304, 306, 308, and 310 of FIG. 3. The histogram 412 corresponds to a small PRT not visible in FIG. 3. In examples, the Buckles Number is computed for each PRT previously derived based on rock quality values determined by petrophysical rock type classification techniques (e.g., Winland R35, FZI). For pre-production wells across carbonate reservoirs at irreducible water saturation, the Buckles Number is also the capillary bound fluid volume for each PRT.

In an example, assume the Buckles Number is computed as the product of porosity and S_{WIRR} (assume the well is located at the crest and the reservoir is pre-production, therefore S_{w}=S_{WIRR}). Similar rock types share the same or substantially the same Buckles Number. However, are slight differences between the Buckles Number of a specific rock type due to effects such as height free water level. Accordingly, for each PRT, the average Buckles Number is applied for optimal representation of each PRT.

The bulk volume of water is computed using the Equation (5).

$$B V W = porosity * water saturation \qquad ( 5 )$$

Using the PRT derived from Winland R35, FZI, or other rock classification technique, the mean of the BVW (e.g., Buckles Number) is computed for each PRT for the reservoir according to Equation (6).

$$B V W ( PRTi ) = \frac{1}{n} {\sum }_{k = 1}^{n} ( B V W ( PRTi ) ) \qquad ( 6 )$$

where n is the number of PRTs.

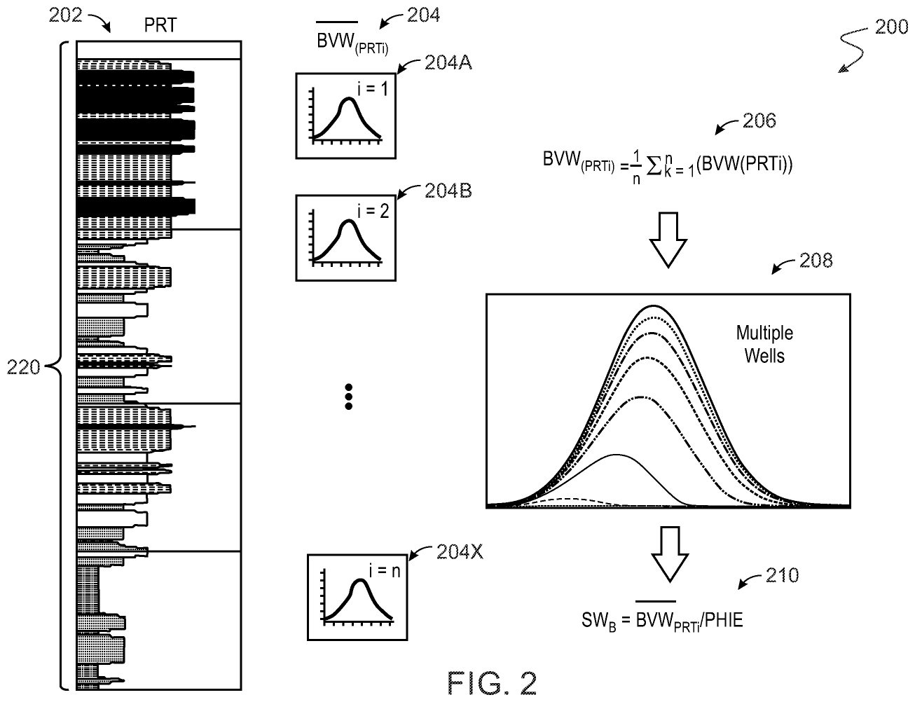

FIG. 2 shows a Buckles Number determination per PRT. As shown in the example of FIG. 2, the PRT types 202 span an interval 220. Each PRT has a corresponding histogram of BVW 204A . . . 204X. The Buckles Number 204 is derived from the histograms of BVW 204A . . . 204X corresponding to each respective PRT of the PRT types 202.

The Buckles Number 204 is determined per PRT for multiple wells 206. By obtaining the Buckles Number using multiple wells, a more precise, accurate statistical representative of the Buckles Number is obtained for each PRT. The Buckles Number derived from multiple wells incorporates effects such as height free water level by using the average value for optimal representation of the Buckles Number for each respective PRT.

At block 208, the resulting robust Buckles Number per PRT is applied to new wells to estimate S_{WB} (S_{WIRR}) using the computed PRT and Porosity. In examples, water saturation in the new wells can be estimated through the Buckles Number, porosity and PRT. A separation between the Buckles Number based water saturation and the resistivity derived water saturation indicates that the water saturation is not at irreducible, which is evidence of water encroachment.

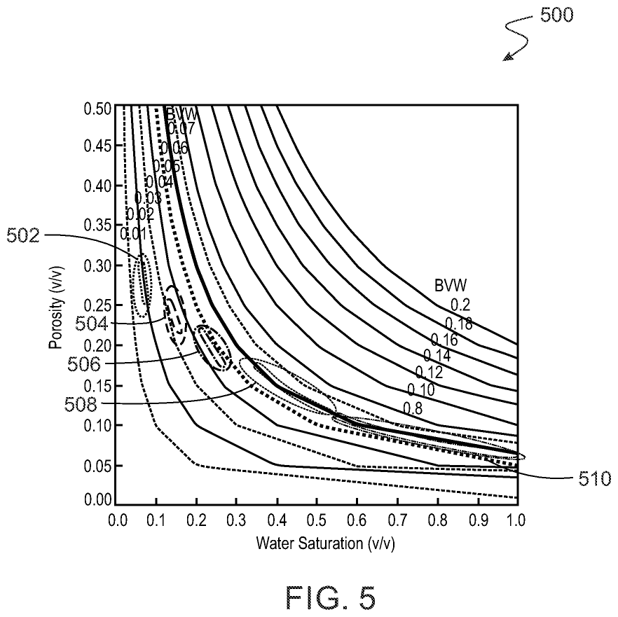

FIG. 5 shows a Buckles plot with mean Buckles Number clusters for respective PRT. The Buckles Number is computed for each petrophysical rock type (PRT) previously derived from petrophysical rock type classification techniques. The clusters 502, 504, 506, 508, and 510, correspond to the PRTs 302, 304, 306, 308, and 310 of FIG. 3. For each respective PRT, contours of equal BVW are drawn on the plot. In examples, points plot on a hyperbolic BVW line where the formation is near immobile water if the points come from a reservoir with consistent pore type and pore geometry. Additionally, in examples, points scatter on a Buckles plot where the formation falls below the top of the transition zone.

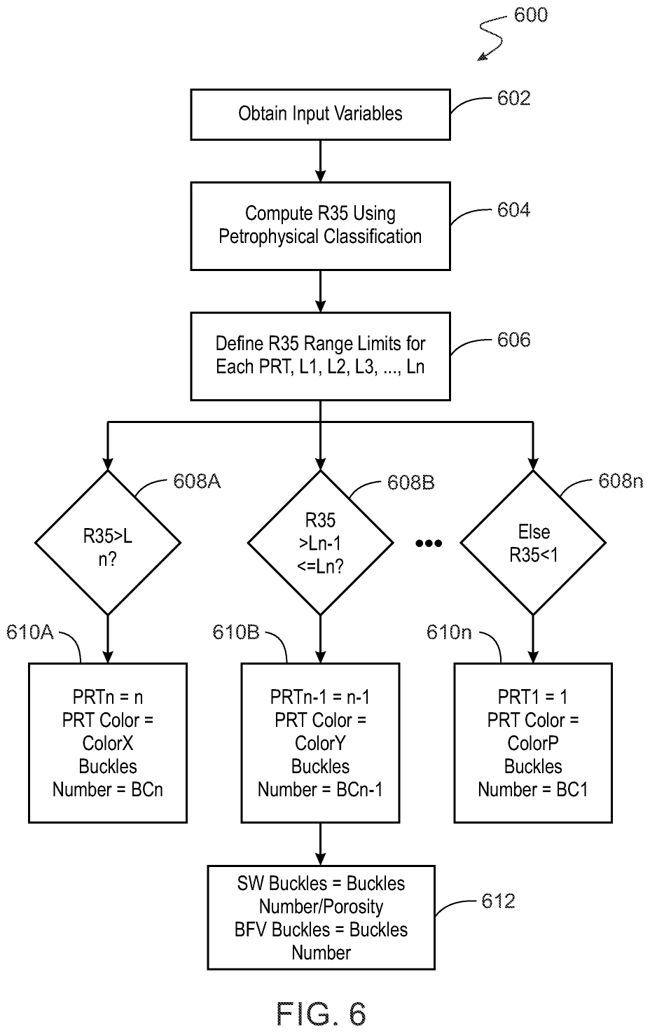

FIG. 6 is a workflow for determining the Buckles Number. In examples, the workflow 600 computes saturation based on the Buckles Number.

At block 602, input variables are obtained. In examples, an algorithm declares and reads the input variables. The input data includes a PRT model as described with respect to FIG. 2 and log data associated with an unseen well.

At block 604, a petrophysical rock type classification of an unseen well is performed. In examples, the petrophysical rock type classification is determined according to the Winland R35, or FZI equations. A rock quality value is generated based on porosity and permeability at various depths of the unseen well.

At block 606, rock property range limits (e.g., rock types) are defined for each PRT of the well. In some embodiments, rock property range limits are defined by the PRT model as described with respect to FIG. 2. In examples, when Winland R35 is used for the petrophysical rock type classification, R35 rock property range limits are defined for each PRT n, L1, L2, L3, . . . , Ln.

The rock quality value is evaluated to assign a PRT-type and Buckles Number (previously computed from the petrophysical rock type classification of the PRT model). For each PRT, the respective rock quality value is compared to the rock property range limit defined for each PRT of the well. Based on the comparison, each rock quality value (e.g., FZI or R35) is assigned a predetermined PRT identified by a number 1−n, wherein each PRT is associated with a color. In examples, the color is used for identification of each respective PRT when plotted in a log or on a graph. Additionally, each PRT is associated with a Buckles Number.

In the example of FIG. 6, at a first decision block 608A, if the rock quality value is greater than range limit Lₙ, then the workflow continues to block 610A. At block 610A, the respective PRT type PRTₙ is equal to n, the PRT color is equal to colorX, and the Buckles Number is equal to BCₙ. At a second decision block 608B, if the rock quality value is greater than range limit Lₙ₋₁ and Lₙ₋₁ is less than or equal to Lₙ, then the workflow continues to block 610B. At block 610B, the respective PRT type PRTₙ₋₁ is equal to n-1, the PRT color is equal to colorY, and the Buckles Number is equal to BCₙ₋₁. The rock quality value is evaluated until at least one of a range limit Lₙ . . . . L₁ is satisfied. Accordingly, at a last decision block 608n, if the rock quality value is less than range limit L₁, then the workflow continues to block 610n. At block 610n, the respective PRT type PRT₁ is equal to 1, the PRT color is equal to colorP, and the Buckles Number is equal to BC₁. In this manner, each rock quality value of the unseen well is assigned a PRT by iteratively comparing the rock quality value to the defined range limits until the rock quality value satisfies at least one range limit.

At block 612, the Buckles Number derived water saturation (S_{w}) is computed as the Buckles Number divided by porosity. Additionally, the bound fluid volume (BFV) is equal to the Buckles Number. In some embodiments, a separation between the Buckles Number based water saturation and the resistivity derived water saturation indicates that the water saturation is not at irreducible, which is evidence of water encroachment. Additionally, in some embodiments bound fluid is calculated simultaneously with water encroachment.

By estimating water encroachment, the present techniques reduce or eliminate uncertainty in calculated water saturation in fields where there is water encroachment. Further the present techniques enable estimation of bound fluid (e.g, BFV) where it is not technically or operationally possible to run NMR logs. In some embodiments, the estimated water encroachment and/or bound fluid volume is used to characterize the reservoir. Characterizing the reservoir provides accurate and detailed information about the reservoir's properties, such as its size, shape, rock properties, fluid content, and fluid flow behavior. In examples, these properties are used to guide further drilling operations at the reservoir.

In some embodiments, the estimated water encroachment and/or bound fluid volume is used to model a reservoir. In examples, the reservoir model is used to simulate the flow of fluids in the reservoir to predict how water will encroach into the reservoir over time. The predictions are used to guide further drilling operations at the reservoir. Additionally, in some embodiments, the estimated water encroachment and/or bound fluid volume is used in the monitoring and management of a reservoir. Management strategies are implemented at the reservoir to minimize the impact of water on production. In examples, management strategies include adjusting production rates, injecting water or other fluids into the reservoir to maintain pressure, or implementing water treatment or disposal programs at the reservoir.

In examples, the workflow 600 is implemented by the following algorithm. Although Winland R35 is described as the petrophysical rock type classification technique, any petrophysical rock type classification technique can be used.

Algorithm I: n PRT types (PRTi ...
PRTn) derived from Winland R35

```
Start of Algorithm
  Declare variables
  i = 1 to n
  Compute R35 Using Winland equation
  LogR35= 0.732 + 0.588*log(Permeability) − 0.864*Log(Porosity)
  Define the R35 range limits for each PRT
  Lᵢ = Aᵢ for PRTᵢ
  .
  .
  .
  Lₙ = Aₙ for PRTₙ
  if R35 is greater than Lₙ
    PRTₙ = n
    PRT color = colorX
    Buckles Number = BCₙ
    SW buckles = Buckles Number / porosity
  Elseif R35 less than or equal to Lₙ and R35 is greater than Lₙ₋₁
    PRTₙ₋₁ = n−1
    PRT color = colorY
    Buckles Number = BCₙ₋₁
    SW buckles = Buckles Number / porosity
  .
  .
  .
  Else
    PRT₁ = 1
    PRT color = ColorP
    Buckles Number = BC₁
    SW buckles = Buckles Number / porosity
End of algorithm
```

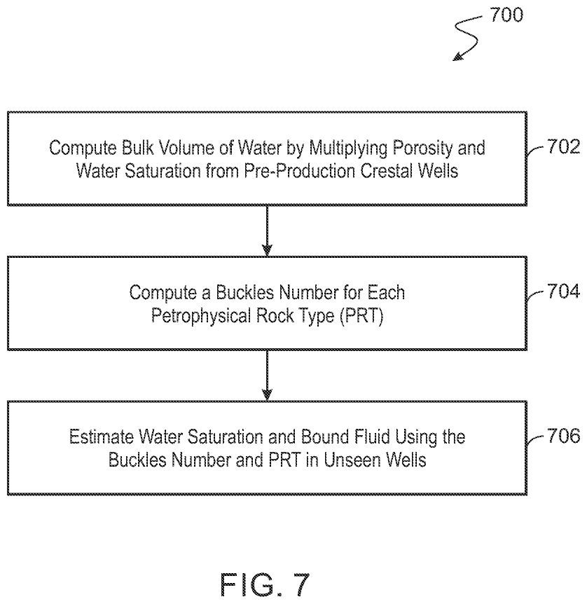

FIG. 7 is a process flow diagram of a process 700 for the estimation of water encroachment and bound fluid in the absence of NMR in carbonate reservoirs using petrophysical rock type and Buckles Number.

At block 702, the bulk volume of water (BVW) is computed by multiplying porosity and water saturation from pre-production crestal wells that are at irreducible water saturation. In some embodiments, the water saturation is derived from saturation height function or a water saturation model.

At block 704, a mean BVW/Buckles Number is computed for each petrophysical rock type (PRT). The Buckles Number is computed for multiple wells in a carbonate reservoir.

At block 706, water saturation in unseen wells (e.g., new) is estimated using the Buckles Number, porosity (from porosity logs) and PRT. In some embodiments, a PRT model is used to determine rock quality values at various depths of the unseen wells. The rock quality values are used to determine the PRT present in the unseen well by comparing the rock quality values to predetermined rock property range limits. Each PRT is associated with a Buckles Number, and the respective Buckles Number is used to determine a Buckles Number-derived water saturation for the unseen wells. In some embodiments, the separation between the Buckles-estimated water saturation and the resistivity derived water saturation shows that the saturation is not at irreducible, indicating evidence of water encroachment. Accordingly, in some embodiments water encroachment and the bound fluid volume are simultaneously calculated based on the estimated water saturation. Additionally, in some embodiments, the bound fluid for the new wells is predicted in the absence of NMR data using the PRT and the Buckles number for each respective PRT.

The present techniques enable the use of petrophysical interpretations (porosity and saturation) from open hole logs integrated with petrophysical rock type interpretation to estimate water saturation. Uneven water encroachment in different rock types is estimated along with bound fluid volume in the absence of Nuclear Magnetic Resonance (NMR) logs. In some embodiments, as hydrocarbons are produced, a differential pressure causes the water encroachment from oil/gas/water contact. Large packets of hydrocarbons can be bypassed and left behind the encroaching water resulting in the reduction of ultimate hydrocarbon recovery. For example, this occurs in in heterogeneous reservoirs where pressure depletion is not uniform thus the hydrocarbon sweep is spatially distributed. The integration of PRT in water encroachment analysis enables a precise definition of water encroachment. Different rock types that have not been encroached above the current oil water contact are identified, and creating opportunity for infill well placement. Additionally, the present techniques enable an accurate calculation of water saturation in reservoirs undergoing water injection, and an estimate of bound fluid in wells where it is not technically or operationally possible to run NMR logs. Moreover, the present techniques enable an optimization of a formation fluid sampling program (e.g., planned activities that aim to obtain representative samples of fluids such as oil, gas, and water from a hydrocarbon reservoir), leading to lower logging costs. For example, if the computed total bulk volume of water from resistivity derived saturation in the unseen well is higher than the bound fluid volume calculated from the Buckles Number and PRT of the unseen well, this means there is mobile water resulting from water encroachment due to production. Thus, the sampling program can be optimized to avoid taking water samples if the sampling objective is to obtain only hydrocarbon samples. This optimization can lead to a significant cost savings.

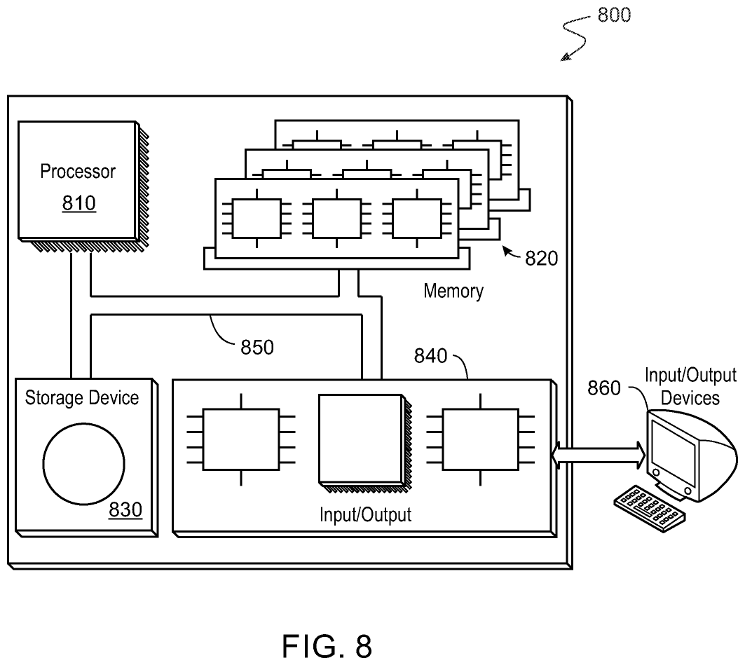

FIG. 8 is a schematic illustration of an example controller 800 (or control system) for the estimation of water encroachment and bound fluid in the absence of NMR in carbonate reservoirs using petrophysical rock type and Buckles Number according to the present disclosure. For example, the controller 800 may be operable according to the workflow 100 of FIG. 1, the workflow 600 of FIG. 6, or the process 700 of FIG. 7. The controller 800 is intended to include various forms of digital computers, such as printed circuit boards (PCB), processors, digital circuitry, or otherwise parts of a system for supply chain alert management. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 800 includes a processor 810, a memory 820, a storage device 830, and an input/output interface 840 communicatively coupled with input/output devices 860 (for example, displays, keyboards, measurement devices, sensors, valves, pumps). Each of the components 810, 820, 830, and 840 are interconnected using a system bus 850. The processor 810 is capable of processing instructions for execution within the controller 800. The processor may be designed using any of a number of architectures. For example, the processor 810 may be a CISC (Complex Instruction Set Computers) processor, a RISC (Reduced Instruction Set Computer) processor, or a MISC (Minimal Instruction Set Computer) processor.

In one implementation, the processor 810 is a single-threaded processor. In another implementation, the processor 810 is a multi-threaded processor. The processor 810 is capable of processing instructions stored in the memory 820 or on the storage device 830 to display graphical information for a user interface on the input/output interface 840.

The memory 820 stores information within the controller 800. In one implementation, the memory 820 is a computer-readable medium. In one implementation, the memory 820 is a volatile memory unit. In another implementation, the memory 820 is a nonvolatile memory unit.

The storage device 830 is capable of providing mass storage for the controller 800. In one implementation, the storage device 830 is a computer-readable medium. In various different implementations, the storage device 830 may be a floppy disk device, a hard disk device, an optical disk device, or a tape device.

The input/output interface 840 provides input/output operations for the controller 800. In one implementation, the input/output devices 860 includes a keyboard and/or pointing device. In another implementation, the input/output devices 860 includes a display unit for displaying graphical user interfaces.

There can be any number of controllers 800 associated with, or external to, a computer system containing controller 800, with each controller 800 communicating over a network. Further, the terms “client,” “user,” and other appropriate terminology can be used interchangeably, as appropriate, without departing from the scope of the present disclosure. Moreover, the present disclosure contemplates that many users can use one controller 800 and one user can use multiple controllers 800.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware—and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship. Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method: a non-transitory, computer-readable medium storing computer-readable instructions to perform the computer-implemented method: and a computer system comprising a computer memory interoperably coupled with a hardware processor configured to perform the computer-implemented method or the instructions stored on the non-transitory, computer-readable medium.

Particular embodiments of the subject matter have been described. Other embodiments are within the scope of the following claims. For example, the actions recited in the claims can be performed in a different order and still achieve desirable results. As one example, some processes depicted in the accompanying figures do not necessarily require the particular order shown, or sequential order, to achieve desirable results.

What is claimed is:

1. A computer-implemented method for estimating water encroachment and bound fluid, the method comprising:
computing, using at least one hardware processor, a bulk volume of water (BVW) per petrophysical rock type (PRT) by multiplying porosity and water saturation for respective PRT types across multiple wells;
computing, using the at least one hardware processor, a Buckles Number for the respective PRT types across multiple wells based on respective BVW; and
estimating, using the at least one hardware processor, water saturation in unseen wells using the Buckles Number, porosity, and PRT, and simultaneously estimating bound fluid for the unseen wells using the PRT and the Buckles Number per PRT.

2. The computer implemented method of claim 1, wherein the water saturation is calculated using a water saturation model or a saturation height function.

3. The computer implemented method of claim 1, wherein PRT types are determined using a rock quality value generated by a petrophysical rock type classification model.

4. The computer implemented method claim 3, wherein the rock quality value is associated with petrophysical rock types according to at least one range limit applied to the PRT.

5. The computer implemented method claim 3, wherein the petrophysical rock type classification model is a Flow Zone Indicator or Winland R35.

6. The computer implemented method of claim 1, wherein the Buckles Number is calculated from a histogram of the BVW per PRT type for the multiple wells.

7. The computer implemented method of claim 1, wherein the porosity for the unseen wells is obtained from log data.

8. An apparatus comprising a non-transitory, computer readable, storage medium that stores instructions that, when executed by at least one processor, cause the at least one processor to perform operations comprising:
computing a bulk volume of water (BVW) per petrophysical rock type (PRT) type by multiplying porosity and water saturation for respective PRT types across multiple wells;
computing a Buckles Number for the respective PRT types across multiple wells based on respective BVW; and
estimating water saturation in unseen wells using the Buckles Number, porosity, and PRT, and simultaneously estimating bound fluid for the unseen wells using the PRT and the Buckles Number per PRT.

9. The apparatus of claim 8, wherein the water saturation is calculated using a water saturation model or a saturation height function.

10. The apparatus of claim 8, wherein PRT types are determined using a rock quality value generated by a petrophysical rock type classification model.

11. The apparatus of claim 10, wherein the rock quality value is associated with petrophysical rock types according to at least one range limit applied to the PRT.

12. The apparatus of claim 10, wherein the petrophysical rock type classification model is a Flow Zone Indicator or Winland R35.

13. The apparatus of claim 8, wherein the Buckles Number is calculated from a histogram of the BVW per PRT type for the multiple wells.

14. The apparatus of claim 8, wherein the porosity for the unseen wells is obtained from log data.

15. A system, comprising:
one or more memory modules;
one or more hardware processors communicably coupled to the one or more memory modules, the one or more hardware processors configured to execute instructions stored on the one or more memory models to perform operations comprising:
computing a bulk volume of water (BVW) per petrophysical rock type (PRT) type by multiplying porosity and water saturation for respective PRT types across multiple wells;
computing a Buckles Number for the respective PRT types across multiple wells based on respective BVW; and
estimating water saturation in unseen wells using the Buckles Number, porosity, and PRT, and simultaneously estimating bound fluid for the unseen wells using the PRT and the Buckles Number per PRT.

16. The system of claim 15, wherein the water saturation is calculated using a water saturation model or a saturation height function.

17. The system of claim 15, wherein PRT types are determined using a rock quality value generated by a petrophysical rock type classification model.

18. The system of claim 17, wherein the rock quality value is associated with petrophysical rock types according to at least one range limit applied to the PRT.

19. The system of claim 17, wherein the petrophysical rock type classification model is a Flow Zone Indicator or Winland R35.

20. The system of claim 15, wherein the Buckles Number is calculated from a histogram of the BVW per PRT type for the multiple wells.
